(19) United States
(12) Patent Application Publication
(10) Pub. No.: US 2009/0228020 A1
(43) Pub. Date: Sep. 10, 2009

(54) IN-SITU GRAFT FENESTRATION

(75) Inventors: Daniel T. Wallace, Santa Cruz, CA (US); Gregory J. Stahler, San Jose, CA (US)

Correspondence Address: VISTA IP LAW GROUP LLP, 12930 Saratoga Avenue, Suite D-2, Saratoga, CA, 95070, US

(73) Assignee: Hansen Medical, Inc., Mountain View, CA (US)

(21) Appl. No.: 12/399,912

(22) Filed: Mar. 6, 2009

Related U.S. Application Data
(60) Provisional application No. 61/068,556, filed on Mar. 6, 2008.

(51) Int. Cl. A61B 19/00

(52) U.S. Cl. 606/130

(57) ABSTRACT

Assemblies, systems, and methods related to in-situ graft fenestration are described. Subsequent to placement of a graft or stent graft into a lumen, such as a blood vessel, a steerable catheter platform is utilized to create fenestrations, or holes, into the material comprising the graft to facilitate flow of fluids, such as blood, out of the holes and into other structures, such as side branch vessels. The catheter platform preferably comprises one or more fenestration elements located distally and configured to controllably create the fenestrations through common graft materials, such as Dacron®. The catheter also may be utilized to size and/or locate side branching structures, confirm fenestration sizes and/or locations, and deploy additional grafts through the fenestrations into other branching structures.

### FIELD OF THE INVENTION

The invention relates generally to remotely controlled medical devices and systems, such as telerobotic surgical systems or manually steerable catheters, and the employment thereof for conducting procedures involving stents and/or stent grafts in body lumens, such as blood vessels. More particularly, this invention relates to systems, apparatuses, and methods for deploying stents and/or stent grafts and creating fenestrations in such devices while they are deployed in situ within body lumens, such as blood vessels, to provide additional flow pathways and/or join with other flow-directing or structural devices.

### BACKGROUND

In certain medical procedures, it is desirable to deploy what is known as a stent or stent graft to structurally support and/or direct flow through a certain passageway, such as blood vessel or other body lumen. Suppliers such as Boston Scientific, Johnson & Johnson, and Medtronic sell stent grafts configured to address disease within the aorta, such as an abdominal aortic aneurysm (“AAA”). Such grafts typically comprise a graft material, such as polytetrafluoroethylene (PTFE) material or the material sold under the tradename “Dacron”®, which may be coupled to a flexible structural frame, typically comprising a metal such as nitinol. Stent grafts typically are constructed to direct flow through one or more lumens defined by the graft material and structural frame, while not allowing substantial flow to pass across the wall of the graft. When a graft needs to be placed in a region where it is desirable to have a certain amount of flow pass across the wall of the graft, a fenestration, or window, may be created in a discrete location of the graft to allow such flow. For example, in a AAA scenario wherein a stent graft is to be placed along a section of the ascending aorta including the takeoff points for the renal arteries, it obviously is not desirable in the typical patient to block flow from the ascending aorta to these renal arteries. One solution is to provide pre-configured fenestrations in a graft which is custom-made for the patient's anatomy. Such a custom-made stent graft may be positioned and deployed to protect the main vessel and also allow flow to the joining vessels. One of the challenges with this approach is that grafts do not always deploy within the actual anatomy as envisioned from preoperative anatomic information; further, the preoperative anatomic information utilized to create the custom graft configuration may not be as accurate as would be desired. Should a pre-configured graft not deploy as expected, it may need to be removed, presenting an undesirable medical scenario.

Another solution is to utilize a graft material that does allow a certain level of flow to cross the wall of the stent-graft construct, thus theoretically enabling placement of a graft right over a joining vessel junction while ensuring that such joining vessel continues to receive flow from the main vessel. One of the challenges with such configurations is that there may be generally more cross-wall leakage than is desirable for a typical disease/graft configuration, and/or inadequate cross-wall flow at key locations near larger vessel takeoffs to address the physiological challenge at hand.

It would be desirable to have a graft configuration that is designed to be deployed into a body lumen and then custom-fenestrated in situ to provide precise, discrete cross-wall flow to other joining lumens in a manner somewhat mimicking what the undiseased anatomy would provide.

### SUMMARY

One embodiment is directed to a robotic system for deploying a medical lumen graft, the system including a remotely steerable flexible instrument having proximal and distal ends and a graft fenestration element coupled to its distal end, the graft fenestration element configured to controllably create a fenestration through a wall of a deployed graft. Also included is a controller in communication with a master input device. Further included is an instrument driver operatively coupled to the controller and the proximal end of the flexible instrument, the instrument driver configured to cause controlled steering movement of the flexible instrument in accordance with input signals received by the controller from the master input device. The graft fenestration element may comprise a resistive element, such as a wire loop, which may comprise a material such as nichrome metal alloy. The graft fenestration element may alternatively comprise a non-resistive discrete heat source, which may be associated with a laser light source or ultrasound transducer source. Further, the graft fenestration element may comprise a mechanical fenestration tip, such as a corkscrew tip or mechanical dilation tip. The flexible guide instrument may define a lumen along its length, which may be configured to provide vacuum to assist in engagement of the guide instrument to other nearby structures. The lumen may be configured to facilitate controllable passage of a branch, or “child”, lumen graft. The system may further comprise a sheath instrument through which the guide instrument may be coaxially disposed. The sheath instrument may comprise a controllably lockable spine structure. The guide instrument lumen may be a working lumen configured to accommodate elongate working instruments, such as needles, guidewires, ablative or fenestrating elements, laser fibers, or the like. The system may further comprise a force sensing apparatus coupled to the instrument driver and configured to sense forces applied distally to instruments inserted through the working lumen. The system may further comprise a localization sensor configured to determine a spatial position of at least a portion of the flexible guide instrument, or other instrument. Such localization sensor may be an electromagnetic sensor, a potential difference sensor, or a fiber-Bragg sensor. An ultrasound transducer may be coupled to the distal end portion of the guide instrument and configured to have a field of view capturing reflected sound information pertinent to a side branch vessel location and/or geometry.

Another embodiment is directed to a method of deploying a lumen graft, wherein subsequent to deploying a parent graft into a parent lumen, one or more locations for fenestration creation in the parent graft are determined utilizing an electromechanically-controlled catheter system comprising a steerable catheter. A fenestration element coupled to the distal tip of the steerable catheter is used to create one or more fenestrations. The fenestration locations may be determined by utilizing a kinematic relationship established for the steerable catheter. Alternatively, such locations may be determined utilizing a localization system, such as one featuring an electromagnetic, potential difference, or fiber-Bragg sensor. Fenestrations may be created by providing current to a resistive element, laser light source, or ultrasound transducer. Fenestrations may also be created by advancing a mechanical fenestration tip, such as one featuring a corkscrew tip or mechanical dilation tip, through a wall of the graft. The method may further comprise utilizing vacuum through a lumen to assist in engaging a catheter structure with adjacent structures, such as the graft or tissues. The method may further comprise confirming the location or size of the one or more fenestrations that have been created. This confirming may comprise using a kinematic relationship established for the steerable catheter, using a localization sensor, such as an electromagnetic, potential difference, or fiber-Bragg localization sensor, using a force sensor, an ultrasound transducer, and/or contrast agent with fluoroscopic imaging. The method may further comprise deploying a child lumen graft through one of the fenestrations, and using an inflatable balloon element to seat such child graft relative to the parent graft. The method may further comprise confirming the location or size of one or more child lumens intersecting with the parent lumen. This confirming may comprise using a kinematic relationship established for the steerable catheter, using a localization sensor, such as an electromagnetic, potential difference, or fiber-Bragg localization sensor, using a force sensor, an ultrasound transducer, and/or contrast agent with fluoroscopic imaging.

### BRIEF DESCRIPTION OF THE DRAWINGS

FIG. 1 illustrates a diagrammatic view of an aorta and related anatomy.

FIGS. 2A and 2B illustrate diagrammatic views of an embodiment of the fenestration system and method whereby contrast agent and fluoroscopy may be utilized to determine geometric and positional aspects of side branching lumens.

FIGS. 3A-3N illustrate diagrammatic views of one embodiment of the fenestration system and method whereby a graft is deployed and then fenestrated in situ.

FIG. 4A illustrates an embodiment wherein a non-resistive fenestration element is utilized to create a fenestration in a graft in situ.

FIG. 4B illustrates an embodiment wherein a bipolar RF fenestration configuration is utilized to create a fenestration in a graft in situ.

FIG. 5 illustrates an embodiment wherein a mechanical fenestration element is utilized to create a fenestration in a graft in situ.

FIG. 6 illustrates a diagrammatic view of an aorta and related anatomy.

FIGS. 7-9 illustrate diagrammatic views of an embodiment of the fenestration system and method whereby contrast agent and fluoroscopy may be utilized to determine geometric and positional aspects of side branching lumens.

FIGS. 10A-10H illustrate diagrammatic views of one embodiment of the fenestration system and method whereby a graft is deployed and then fenestrated in situ.

FIG. 11A illustrates a operating-room-level view of one embodiment of a system configured for executing an in-situ graft fenestration.

FIG. 11B illustrates a diagrammatic view of one embodiment of a system configured for executing an in-situ graft fenestration.

FIG. 11C illustrates a diagrammatic view of one embodiment of a system configured for executing an in-situ graft fenestration.

FIG. 11D illustrates a diagrammatic side view of an instrument assembly configured for executing an in-situ graft fenestration, the assembly including a direct visualization element having a forward-oriented field of view.

FIG. 11E illustrates a diagrammatic side view of an instrument assembly configured for executing an in-situ graft fenestration, the assembly including a direct visualization element having a side-oriented field of view.

FIG. 12A depicts a deployed graft assembly with in-situ fenestrations in an aortic aneurysm anatomical environment.

FIG. 12B depicts a deployed graft assembly with in-situ fenestrations in a bronchial bifurcation anatomical environment.

FIG. 13 illustrates a method for deploying a graft and fenestrating it in situ.

### DETAILED DESCRIPTION

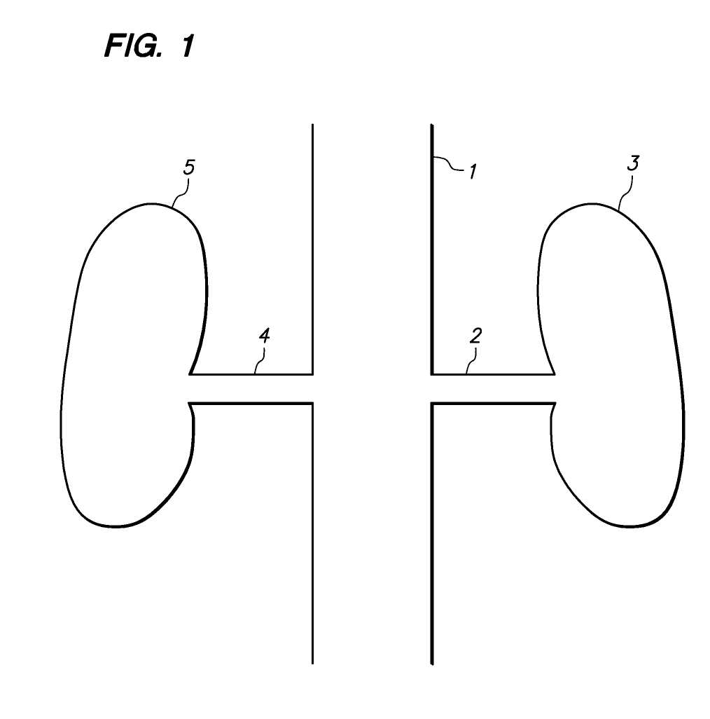

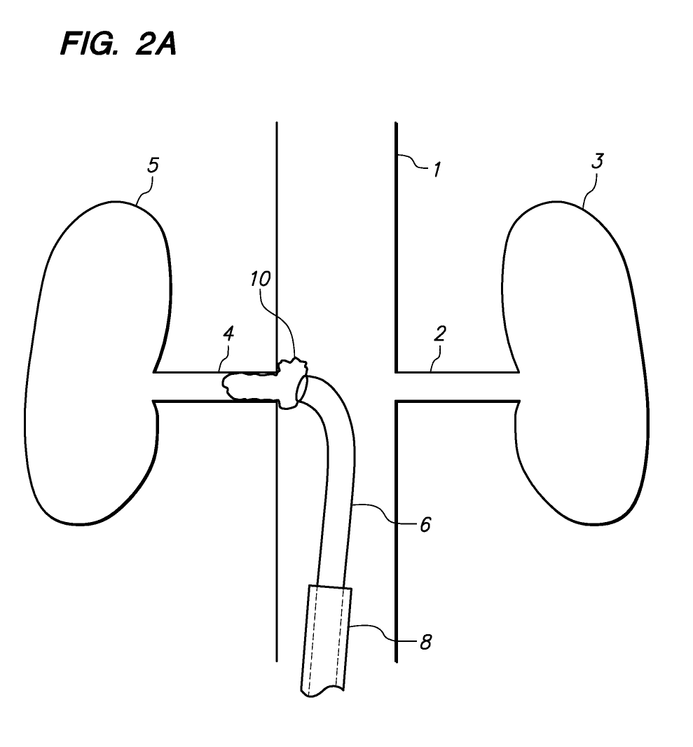

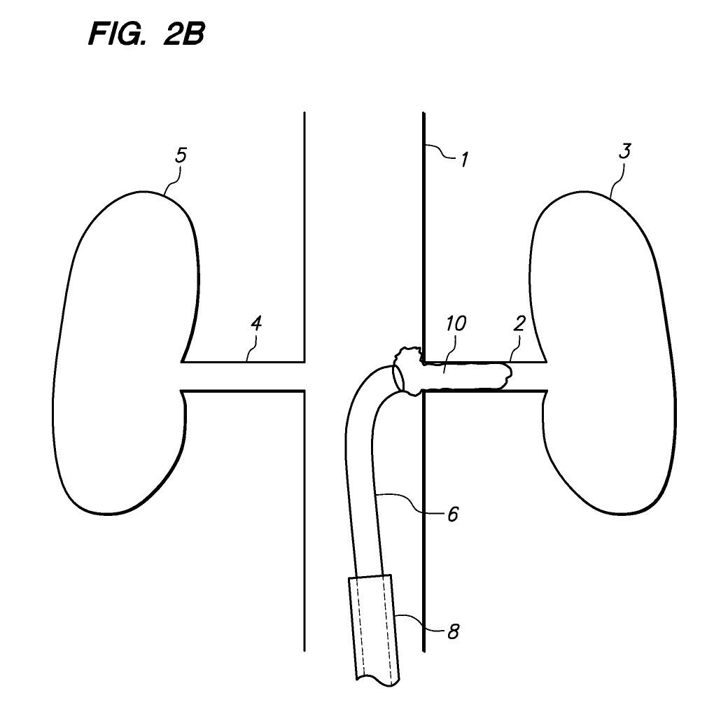

Systems and methods for fenestrating an in situ graft are described herein. Referring to FIG. 1, an exemplary tissue complex comprising the renal arteries (2, 4), kidneys (3, 5) and a portion of the aorta (1) is depicted for illustration purposes. In one embodiment, before deployment of a graft into the aorta, a “main” or “parent” lumen, or either of the renal arteries, each a “branch” or “child” lumen, this tissue complex may be imaged and/or scanned utilizing advanced imaging techniques such as CT, MR, and/or ultrasound, to produce high-resolution voxel images which may be segmented utilizing conventional techniques and turned into triangular mesh models and the like. All of this preferably is accomplished preoperatively. Referring to FIGS. 2A and 2B, injected contrast agent (10) combined with fluoroscopic imaging may be utilized to create images of contrast agent volumes, and these images may be associated with the entrances to the renal arteries (2, 4) from the aorta (1). Such volumes (i.e., of the contrast agent cloud (10)) may also be segmented and turned into models. Utilizing a robotic catheter system comprising, for example, an outer steerable sheath catheter (8) and a coaxially-associated inner sheath catheter (6), such as those described in patent application Ser. Nos. 10/923,660, 10/949,032, 11/073,363, 11/173,812, 11/176,954, 11/179,007, 11/176,598, 11/176,957, 11/185,432, 11/202,925, 11/331,576, 11/418,398, 11/481,433, 11/637,951, 11/640,099, 11/678,001, 11/678,016, 60/919,015, 11/690,116, 60/920,328, 60/925,449, 60/925,472, 60/926,060, 60/927,682, 11/804,585, 60/931,827, 60/934,639, 60/934,688, 60/961,189, 11/762,778, 11/762,779, 60/961,191, 11/829,076, 11/833,969, 60/962,704, 60/964,773, 60/964,195, 11/852,252, 11/906,746, 61/003,008, 11/972,581, 12/022,987, 12/024,883, 12/024,760, 12/024,641, 12/032,626, 12/032,634, 12/032,622, 12/032,639, 12/012,795, and 12/398,763, each of which is incorporated by reference in its entirety into this disclosure, is preferable due to the high level of accurate navigability provided by such a system. Other remotely steerable systems, including those that are manually steered with handles and the like rather than electromechanical instrument driving mechanisms, may also be utilized for the subject procedures, systems, and apparatuses. In the preferred embodiment, the control system of the robotic catheter system is aligned or registered with the preoperatively acquired image data utilizing the fluoroscopy images and interactive fluoroscopy to understand where the instruments are relative to the anatomy. Once the instruments are registered to the image data, the instruments may be “driven” instinctively utilizing the image data, as described in the aforementioned incorporated disclosures. Further, once registered, the catheter system may be utilized to determine locations of branching lumens and other anatomy, through the use of established kinematic relationships pertinent to the catheter instrument set (6, 8), and/or via localization systems, such as those comprising electromagmetic sensors, potential difference sensors, voltage difference sensors, impedance difference sensors, and/or fiber-Bragg sensors.

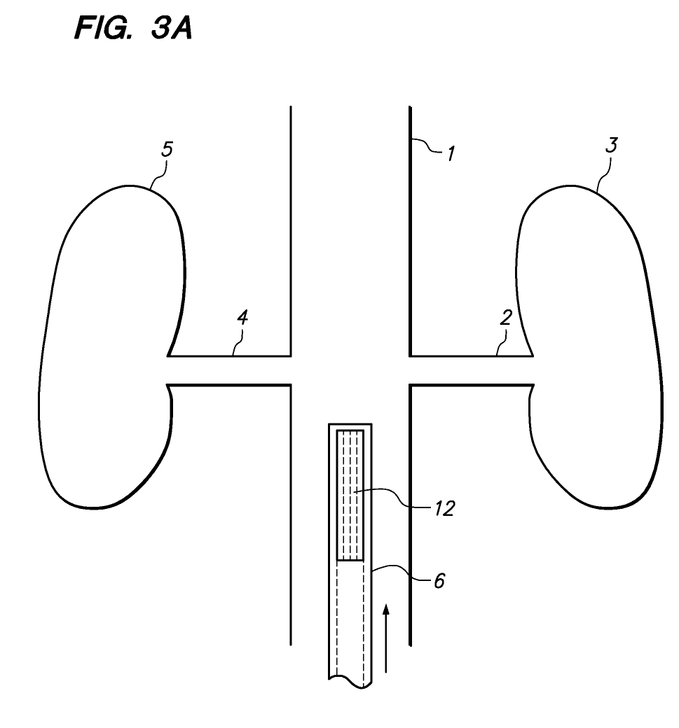

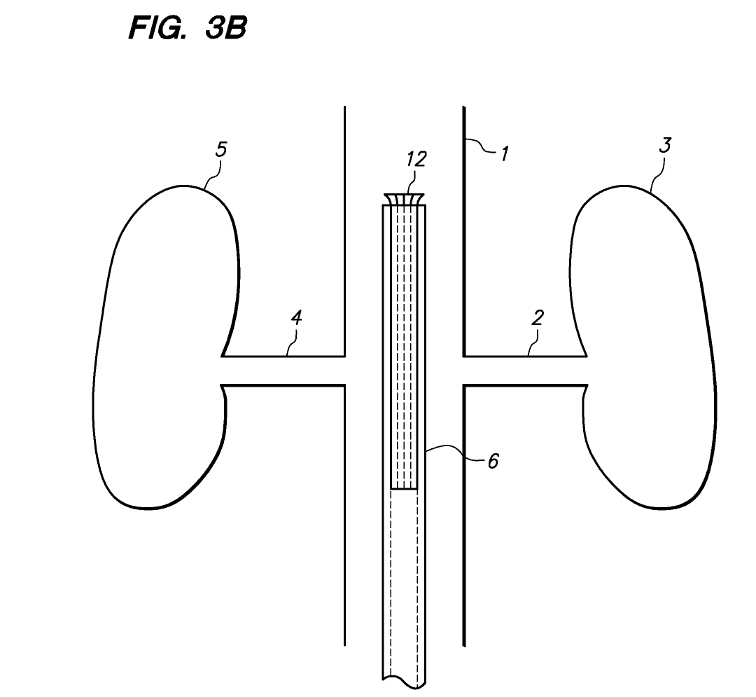

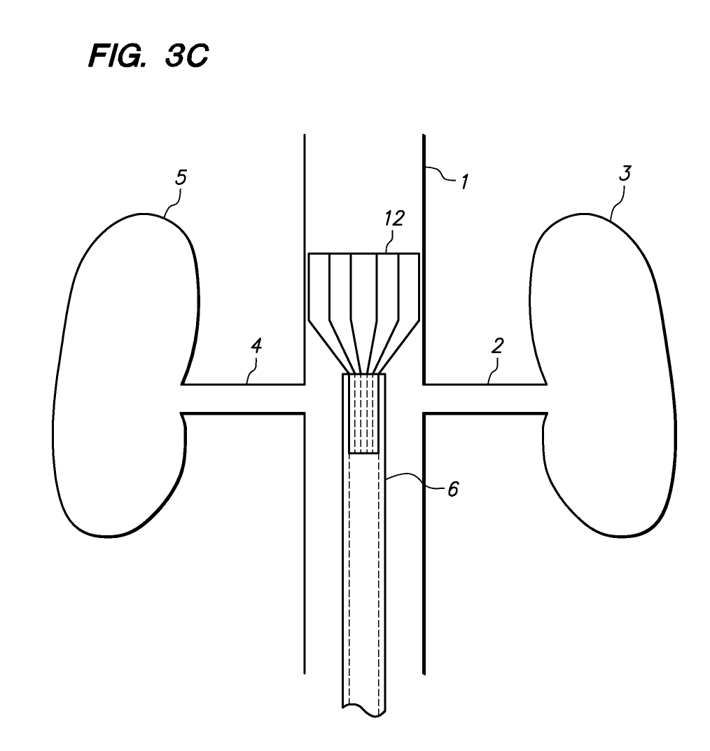

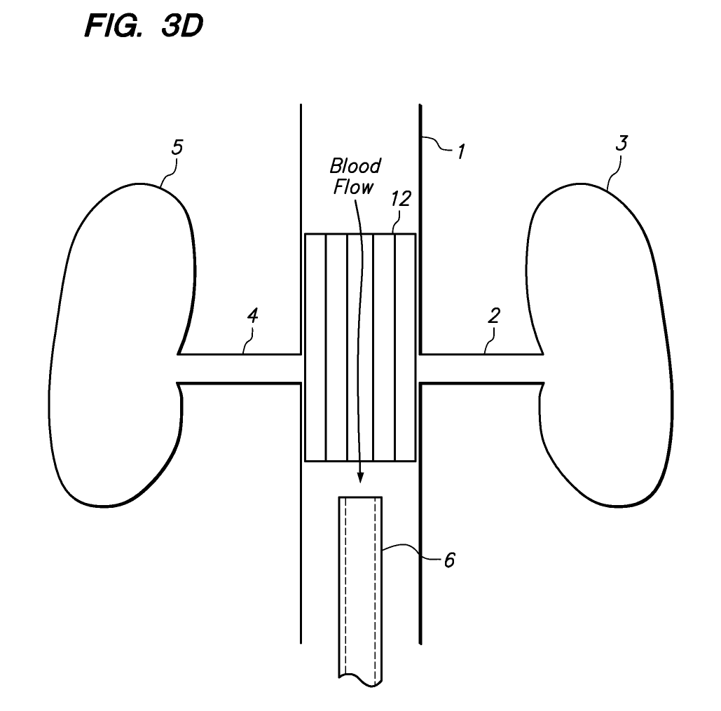

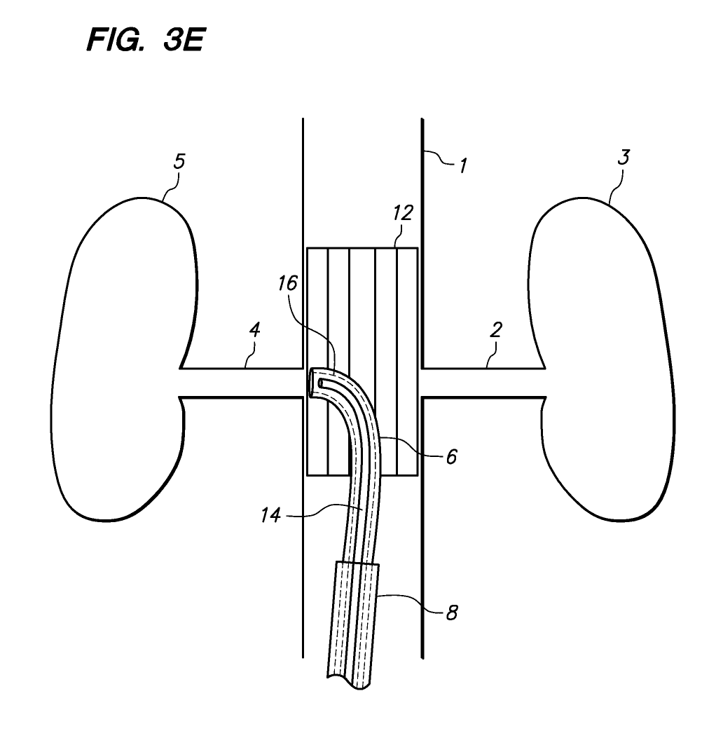

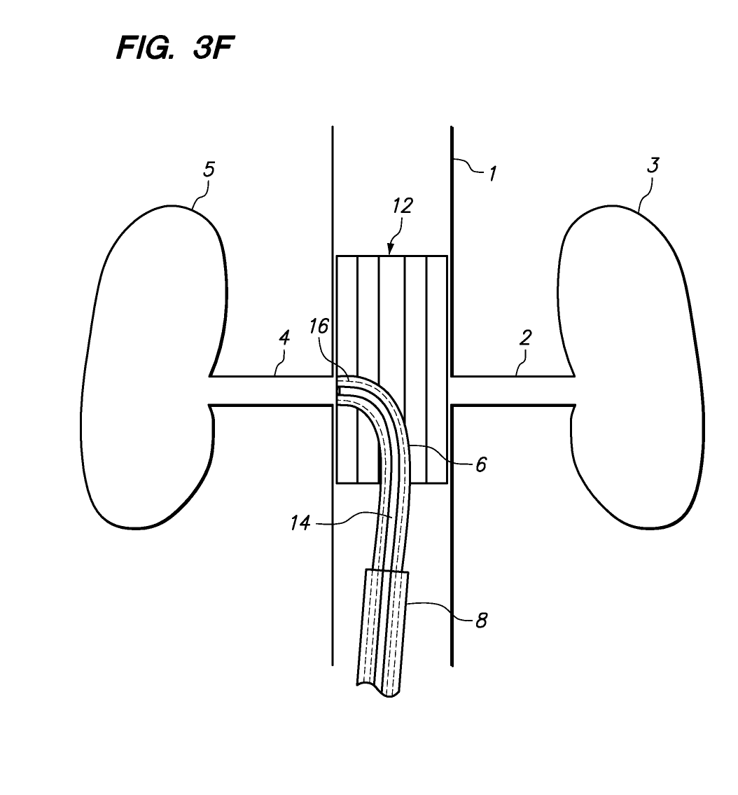

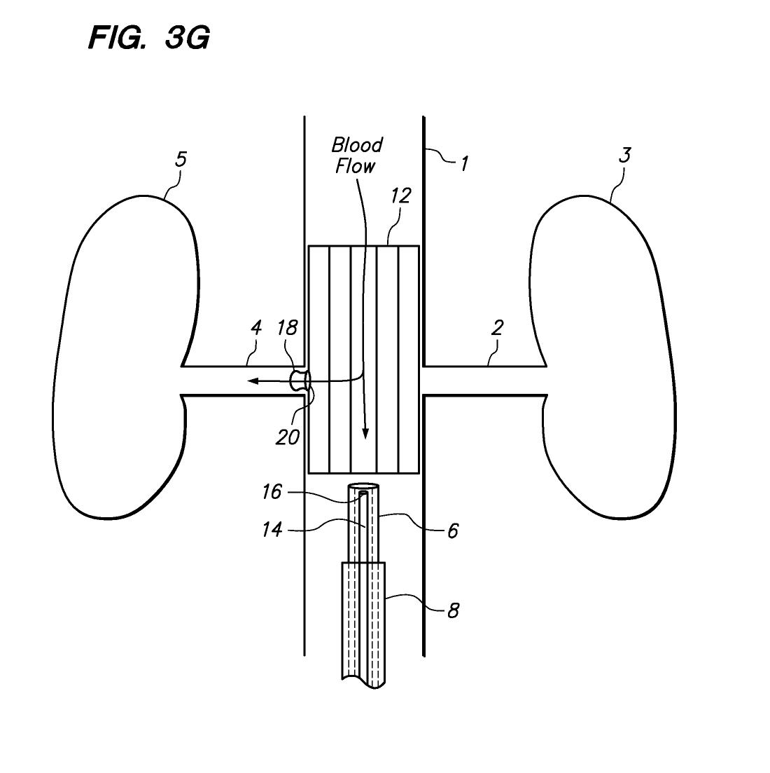

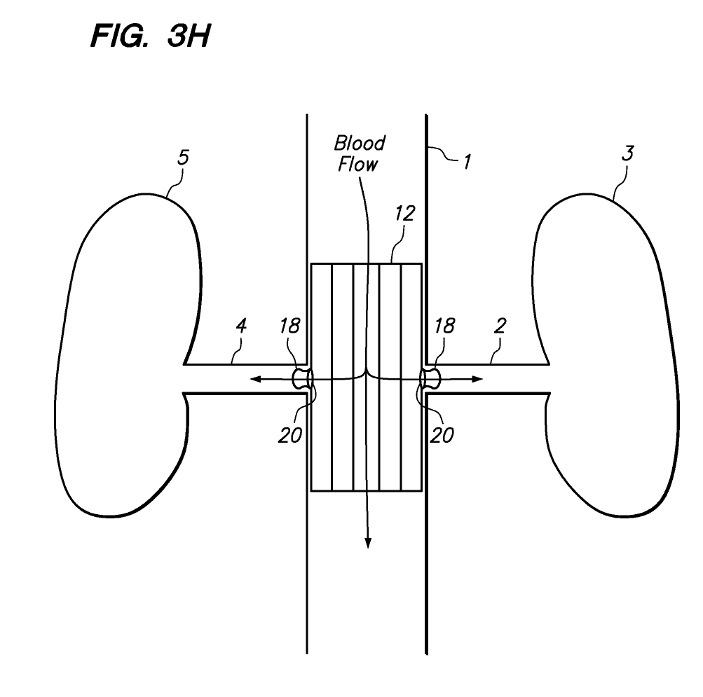

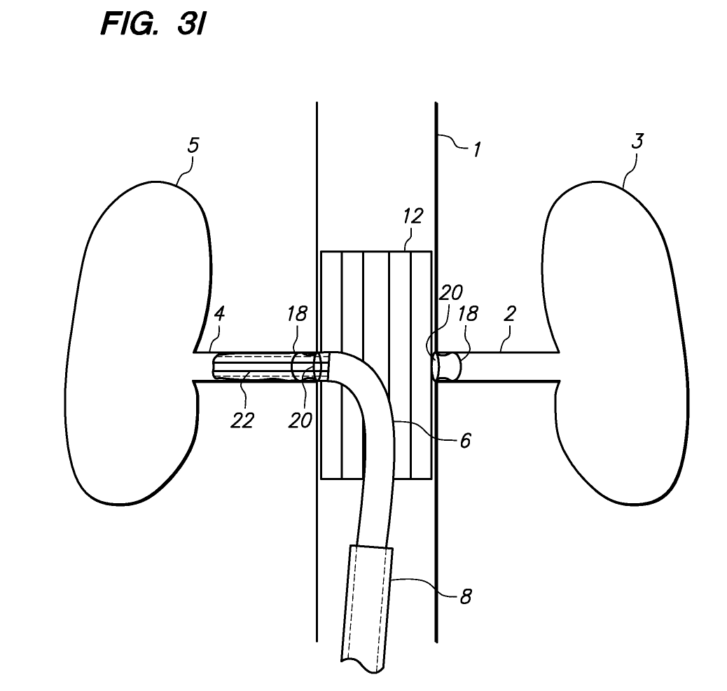

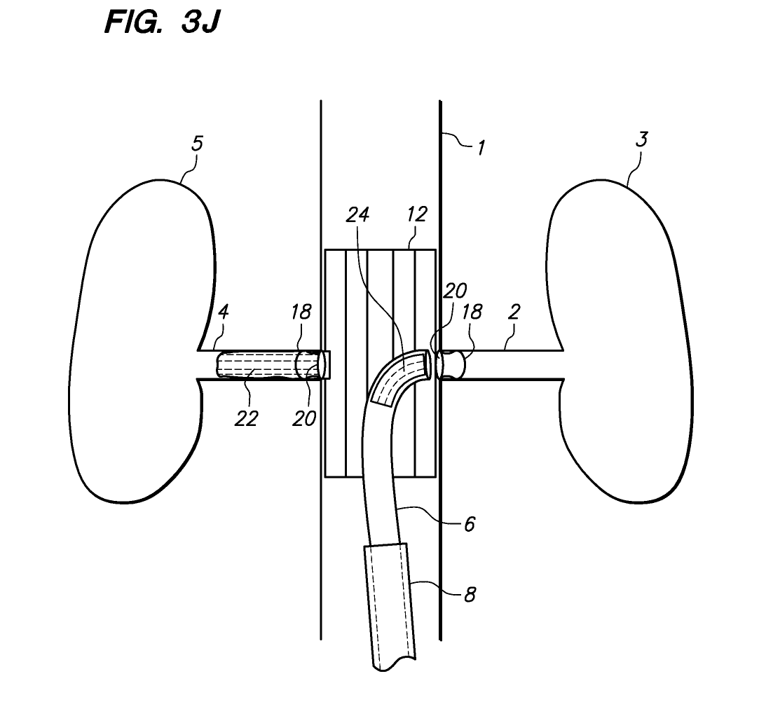

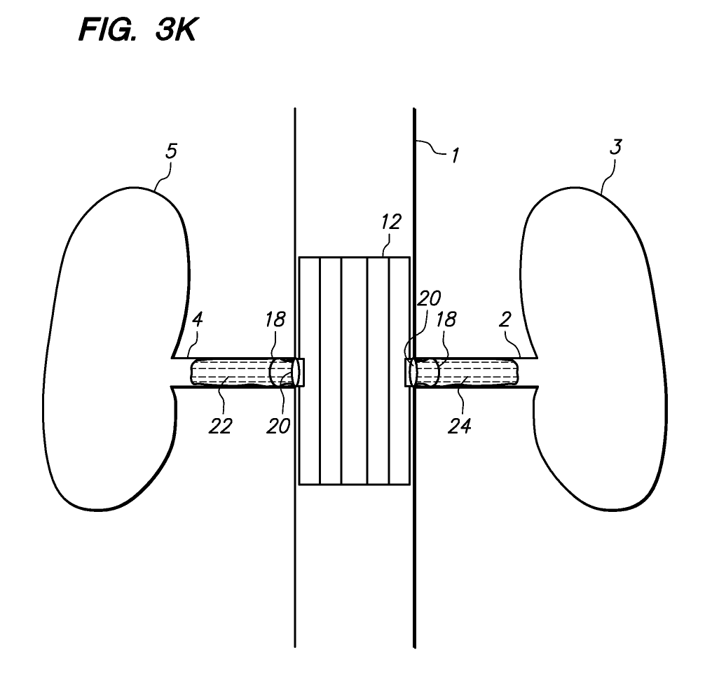

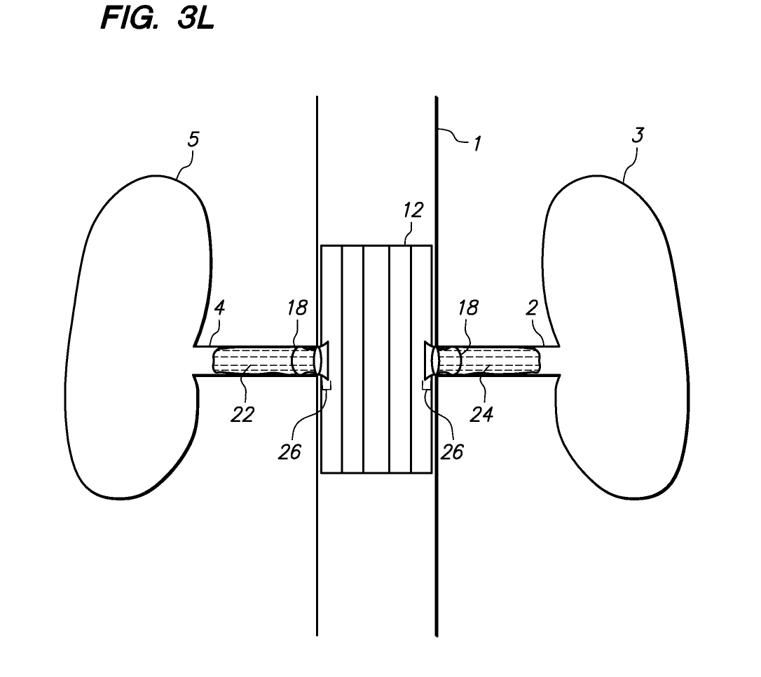

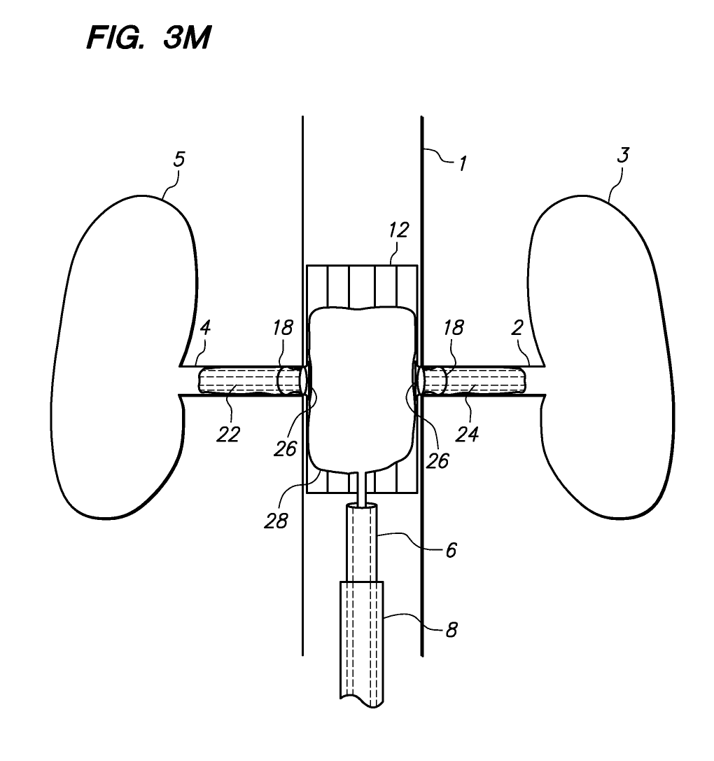

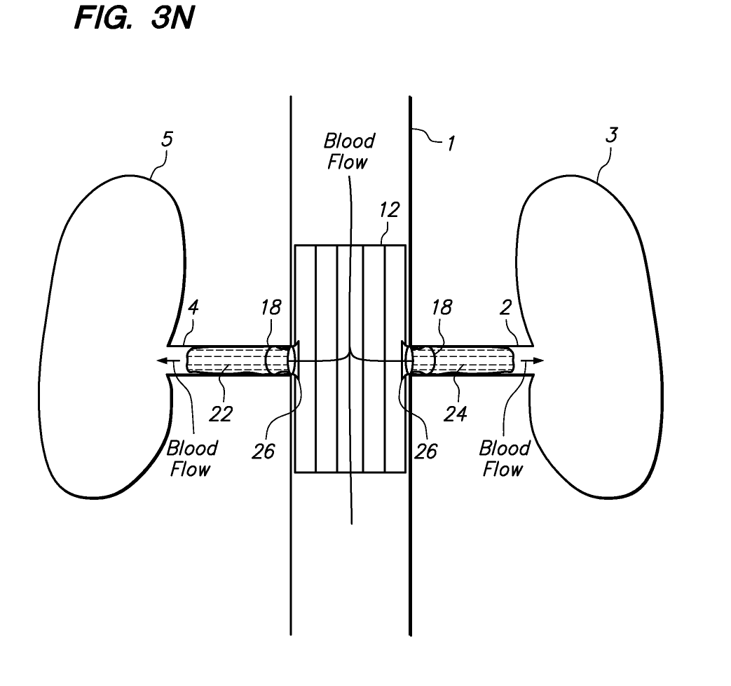

Having determined the locations of the side branching lumens (2, 4) in this example scenario, a parent graft may be placed into the parent lumen (here, the aorta (1)). The parent graft may be reinforced with flexible materials such as nitinol alloy wires, and may be denoted a “stent graft” due to such composite construction. For simplicity, in this example, the parent lumen prosthesis is referred to as a “graft” or “lumen graft” hereinafter, and it should be clear that the graft may or may not include a composite instruction, and may or may not be a stent or stent graft—it may, for example, be an unreinforced vascular or bronchial lumen graft, and may optionally have reinforcement provided by structures other than stent-like reinforcing materials—for example, it may be reinforced utilizing inflatable lumens comprising at least certain portions of the walls of a particular graft variation. Referring to FIG. 3A, the registered instrument system (6) may be navigated up the aorta (1) to deploy a parent graft (12) in a position that spans the openings of to the renal arteries (2, 4). The parent graft (12) is shown in a compressed configuration within the working lumen of a guide instrument (6) in FIG. 3A. Referring to FIG. 3B, the compressed parent graft (12) is pushed out of the guide catheter instrument (6). In this case the parent graft (12) is a self expanding stent graft—but balloon or otherwise expandable prostheses may be utilized as well. FIG. 3C depicts a partially deployed parent graft (12). FIG. 3D depicts a fully deployed parent graft (12) that is directing all of the blood flow inferiorly past the renal arteries (2, 4), which are receiving essentially no flow in this configuration. Referring to FIG. 3E, an instrument assembly (6, 8, 14) is advanced toward the position of the renal artery opening, which is known thanks to the contrast volume that was previously captured and registered to the control system, or thanks to the aforementioned localization and imaging techniques (for example, the locations of the branching lumens may be determined with localization of the distal tip of the catheter, while the sizes of the lumens may be determined using fluoroscopy with contrast, transcutaneous ultrasound, etcetera). A fenestration probe (14) comprises a fenestration element (16) which, as depicted in FIGS. 3F and 3G, may be utilized to cut a hole, or fenestration, in the graft (12) to create a discrete flow channel into the side branching lumen, here the renal artery (4). The fenestration element may comprise a resistive element, such as substantially circular loop of nichrome wire that is selectively electrified (i.e., via the flow of electrical current) by the system operator when cutting of graft material such as Dacron® is desired. Referring to FIG. 3F, the robotic catheter system (6, 8) may be utilized to engage the fenestration element (16) of the fenestration probe (14) to the desired location upon the wall of the graft (12). In one embodiment, a vacuum lumen (not shown) through the inner sheath instrument (6) may be utilized to promote engagement between the inner sheath instrument (6) and graft (12), and thereby assist in the positioning and stabilizing of the fenestration probe (14) during fenestration with the fenestration element (16).

Referring to FIG. 3G, subsequent to fenestration, the in-complete circular loop configuration of the fenestration element (16) is configured to leave behind a flap (18) of graft material that will stay in place. In another embodiment (not shown), a completely circular loop may comprise the fenestration element, and vacuum may be utilized to remove a circular patch of graft material proximally as it becomes loose. With the fenestration completed, blood is free to flow through the fenestration (20), into the renal artery (4), to the kidney (5). As shown in FIG. 3H, this may be done bilaterally. Referring to FIG. 3I, in the event that it is desirable to also place a stent or stent graft (18) into a child or branching lumen such as one or both of the renal arteries, a similar instrument assembly (6, 8) and robotic control system may be utilized to navigate a smaller “child” graft (22) through the pertinent fenestration (20) and into the renal artery (4) as shown. Referring to FIGS. 3J and 3K, this may be conducted bilaterally with another child graft (24). Referring to FIG. 3L, the proximal ends of the child grafts (22, 24) may have flanged geometries (26) to assist in smooth flow and/or prevention of distal child graft migration (i.e., prevention of migration of such grafts toward the kidneys (3, 5) any farther than desired). Referring to FIGS. 3M and 3L, these flanged portions (26) may be compressed into place, and in one embodiment deformed as they are compressed, against the larger graft (12) adjacent the fenestrations (20) with an expandable balloon element (28) or other expandable instrument to seat the flanged portions (26) securely against the larger graft (12) body. As shown in FIG. 3N, blood flow preferably mimics the original anatomy in that it flows through the graft to the rest of the ascending aorta, and also to the kidneys through the fenestrations (20).

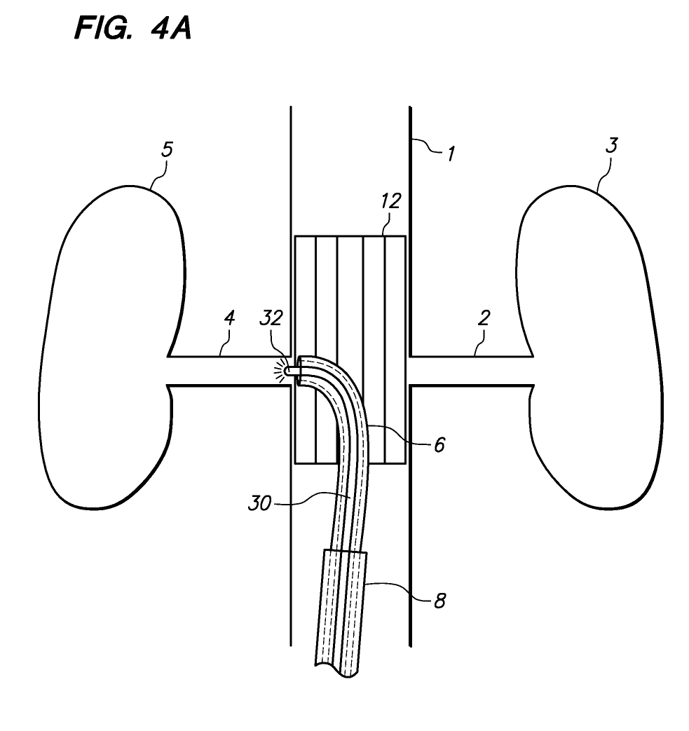

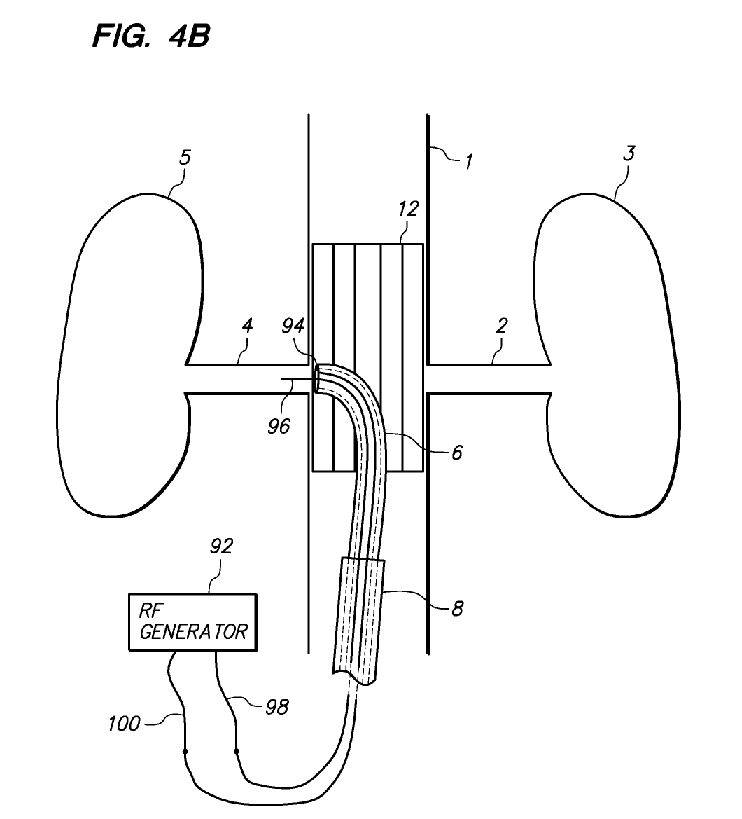

Referring to FIG. 4a, a distal fenestration probe embodiment is depicted wherein an alternative to the cutting loop fenestration element (16) described in reference to FIGS. 3A-3N is depicted. As shown in FIG. 4, fenestrations may also be created using a discrete heating element (32) located at the distal tip of a flexible probe (30). Such heating element my generate heat as a result of its connectivity with a source of current or otherwise electrical actuation. In one embodiment, heat may be generated by passing RF energy to a monopolar electrode. In another embodiment, such as that depicted in FIG. 4B, a bipolar electrode configuration may be utilized. Referring to FIG. 4B, an inner sheath instrument (6) is depicted threaded through the working lumen of an outer sheath instrument (8). A needle probe (96) is threaded through the working lumen of the inner sheath instrument (6) and is electrically coupled proximally with a lead (98) to an RF generator (92). Also electrically coupled to the RF generator (92) by a different lead (100) is a fenestration element (94) coupled to the distal end of the inner sheath instrument (6). In one embodiment, the fenestration element is connected to be an anode and the needle probe (96) tip connected to be a cathode; in another embodiment, the fenestration element is connected to be a cathode and the needle probe (96) tip connected to be an anode. In either of these embodiments, when the RF generator is turned on, current flows between the cathode and anode and create a fenestration in the targeted graft material. Other suitable discrete heating elements comprise laser fibers and related distal terminations, distally-positioned high-intensity ultrasound transducers, and/or one or more resistively-heated blunt geometry heat sinks positioned distally.

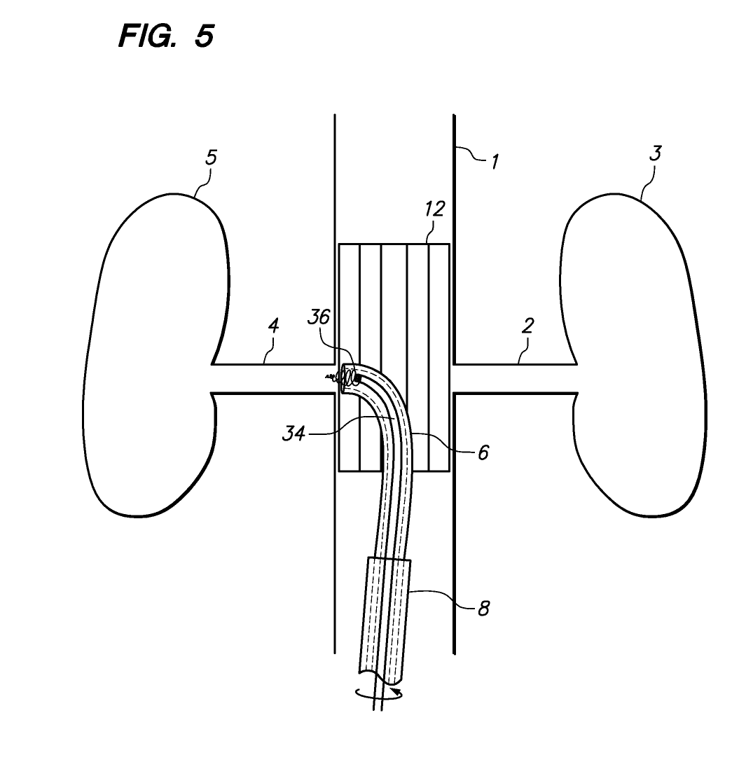

Referring to FIG. 5, a rotatable fenestration probe (34) having a drill bit or corkscrew style distal tip mechanical fenestration element (36) may also be utilized to create fenestrations. Alternatively the distal portion of a fenestration probe embodiment may comprise a simple tapered dilator tip or punch (not shown) configured to pass through the graft wall material and plastically deform it to create a fenestration; such a punch configuration may be operably coupled to a mechanism configured to controllably advance the punch a finite distance with a high-inpulse load upon triggering, similar to the “guillotine” type mechanisms utilized in guillotine type biopsy needles, such as those available from manufacturers such as Egemen International, Inc. In each fenestration variation, vacuum, for example through the working lumen of the smaller catheter (6), may be utilized to engage the graft material to the catheter (6) tip and facilitate fenestration.

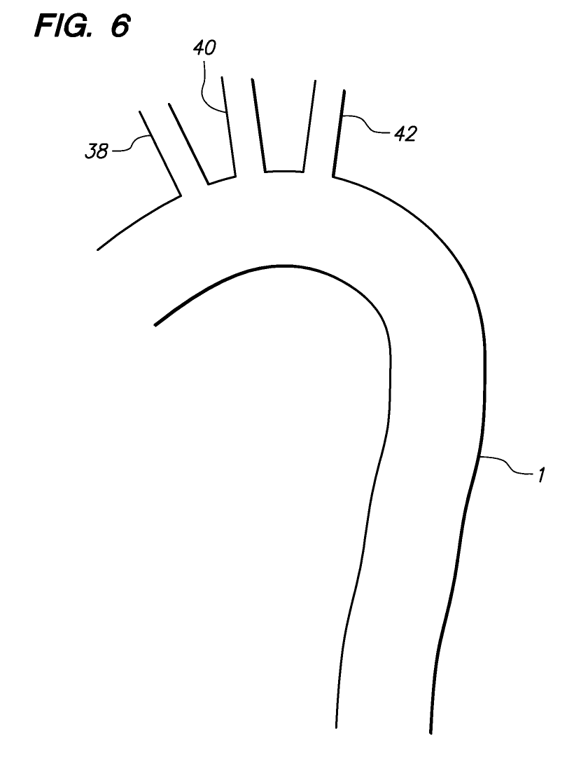

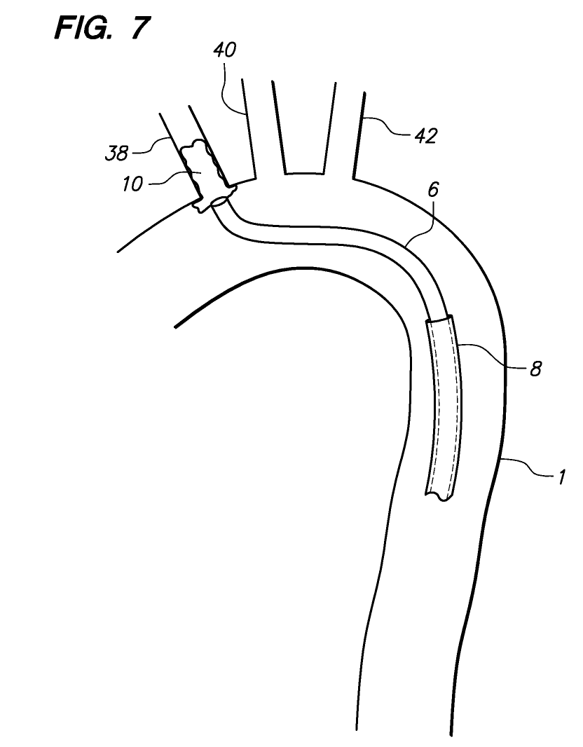

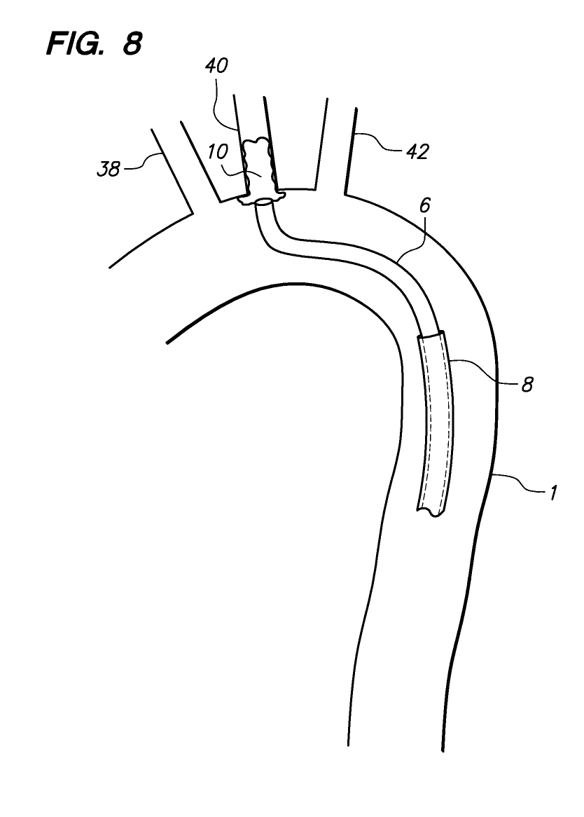

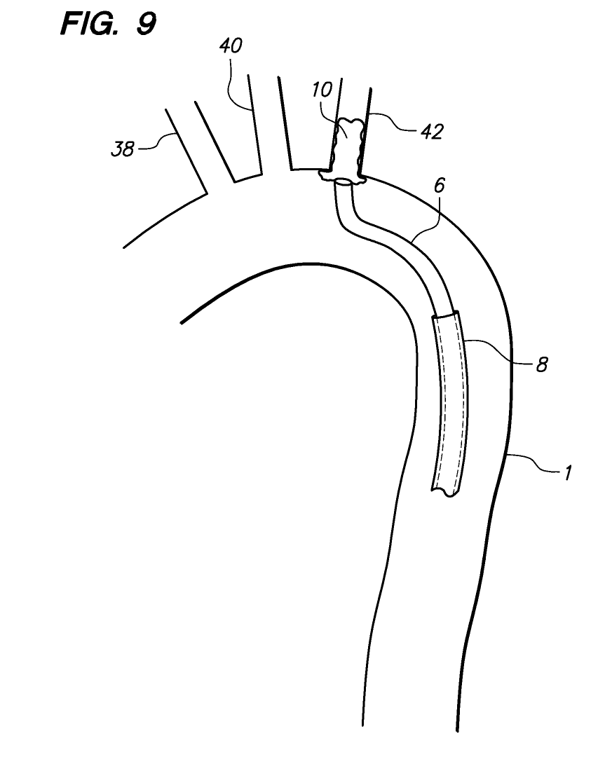

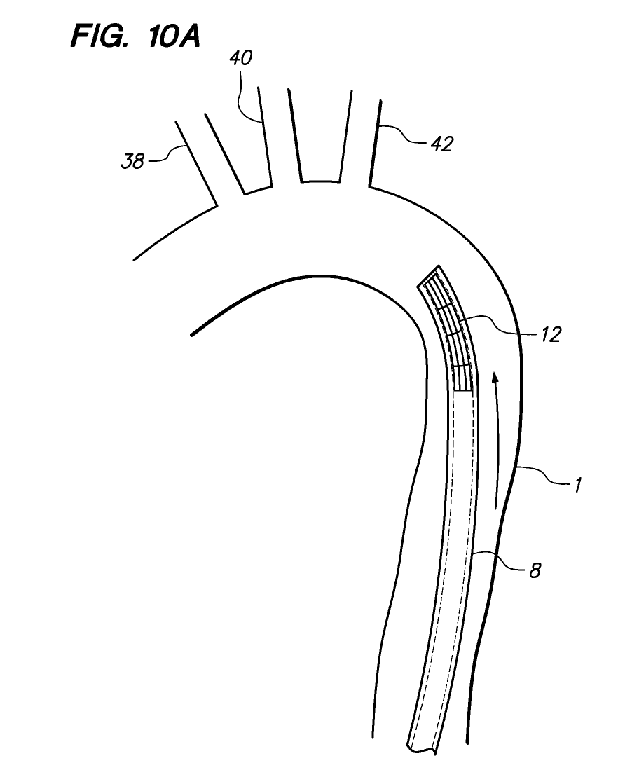

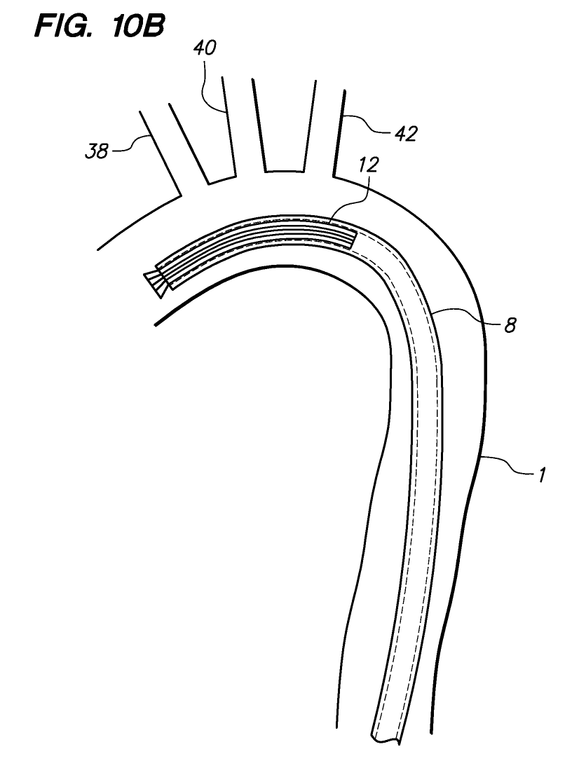

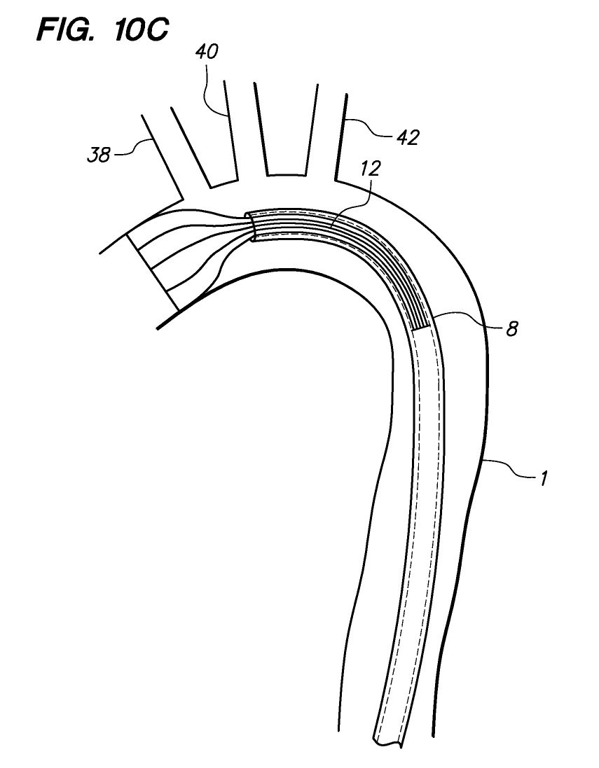

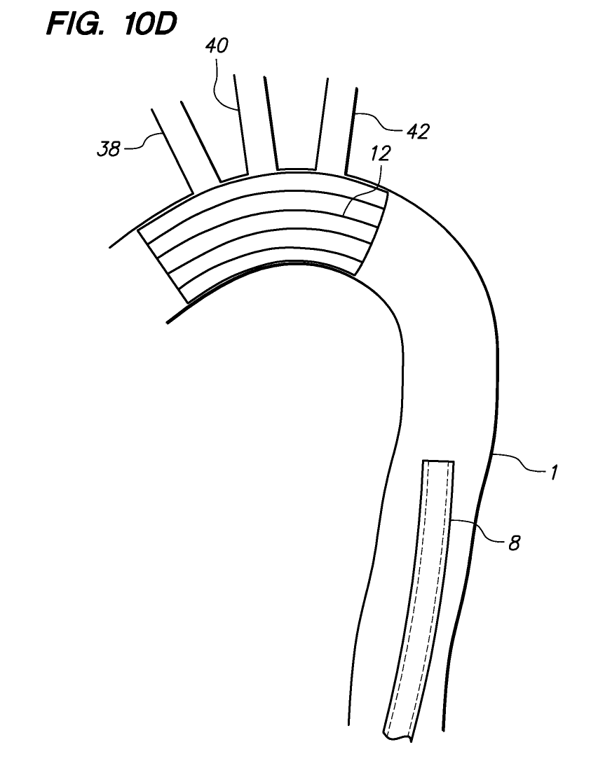

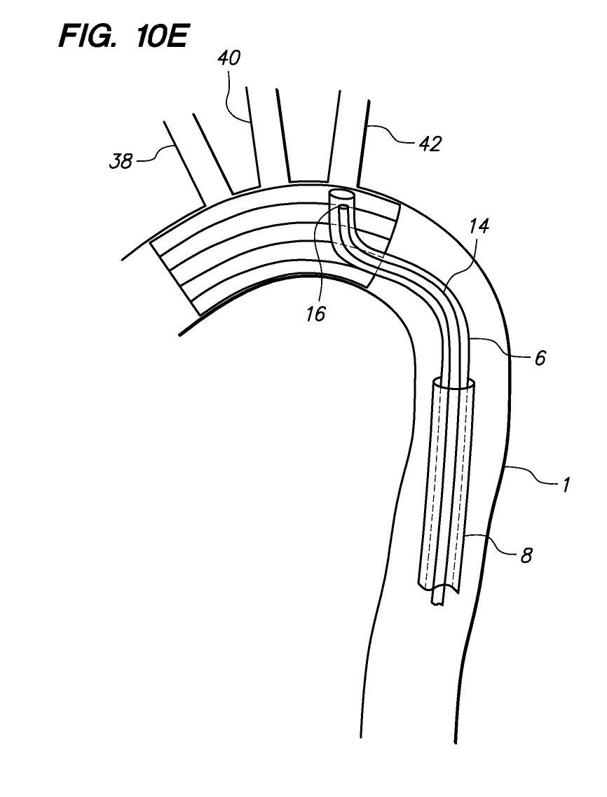

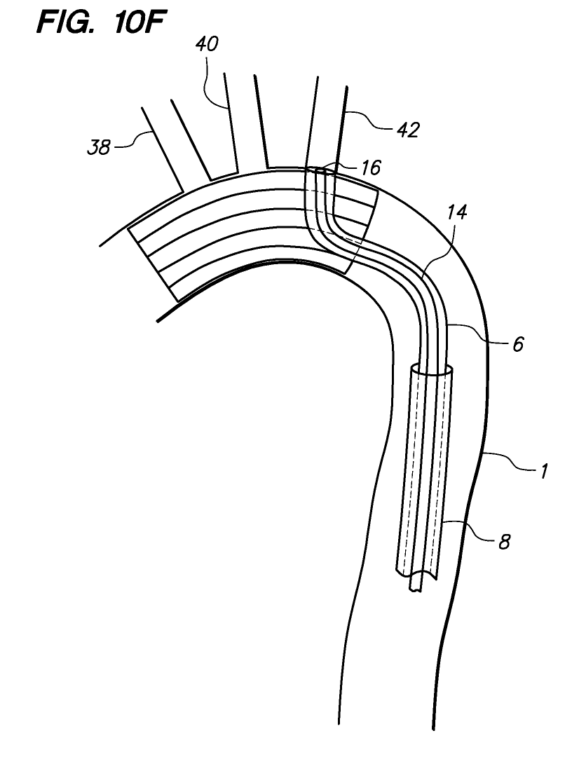

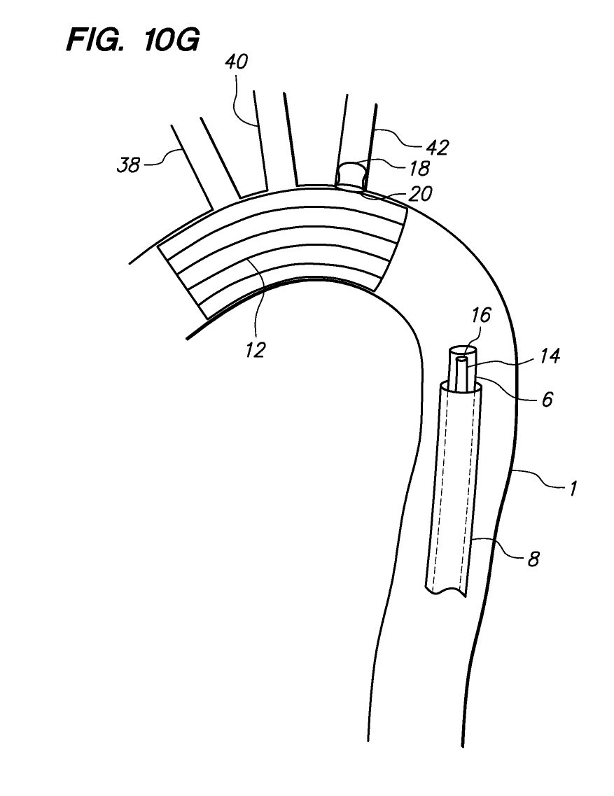

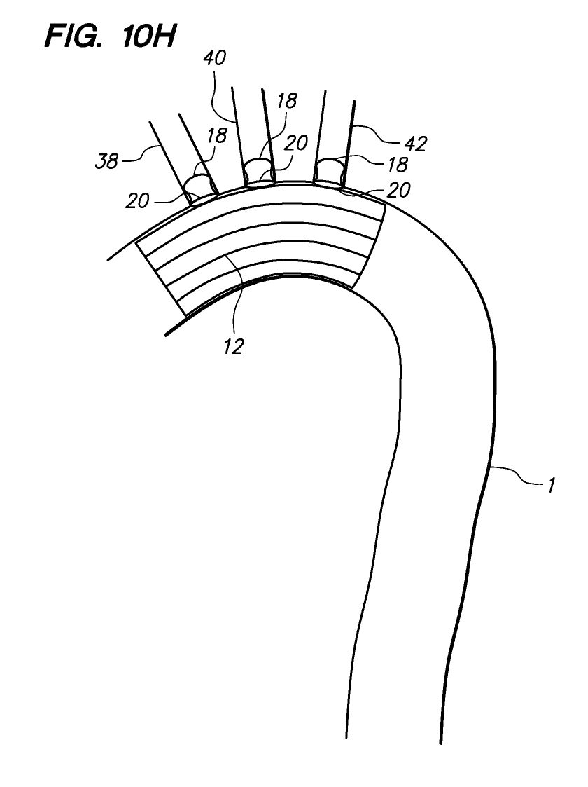

Referring to FIGS. 6-10H, another in-situ graft fenestration is illustrated, this example in the region of the aortic arch. Similar technological issues are encountered and solved by the inventive systems and methods. Referring to FIG. 6, an aorta (1) and branching arteries, such as the brachiocephalic (38), common carotid (40), and left subclavian (42) arteries are depicted. Referring to FIGS. 7, 8, and 9, in a manner similar to that described in relation to FIGS. 2A-2B, the positions and geometries of the branching lumens (38, 40, 42) may be characterized utilizing contrast agent disbursal, fluoroscopy, and/or localization via kinematic and/or localization sensor-based techniques. Referring to FIG. 10A, a compressed parent graft (12) is advanced toward the grafting location in the parent lumen, here the aorta (1). Referring to FIGS. 10B and 10C, the compressed parent graft (12) is pushed out of the delivering catheter device (8). Referring to FIG. 10D, the parent graft (12) is deployed and expanded in place across the aorta (1) and at least partially blocking the side branching arteries (38, 40, 42). In one embodiment, a substantially non-occluding graft material may be utilized to promote at least some flow across the wall of the deployed graft (12) in this position before in-situ fenestration to provide the ultimately desired flow condition. Alternatively, the system may be configured to work very efficiently following deployment of the graft, for example, by virtue of automation options provided with the robotic catheter system described in detail in the aforementioned applications which are incorporated by reference herein. Referring to FIGS. 10E and 10F, an instrument assembly (6, 10,14) is advanced toward the predetermined fenestration locations, which have preferably been determined utilizing techniques such as those described in reference to the above renal grafting scenario. Referring to FIG. 10G, a fenestration (20) and flap (18) are created with the fenestration element (16) of the fenestration probe (14), allowing flow through the first targeted side branching vessel (42). Similarly, the other vessel locations are fenestrated to provide flow to all of the targeted side branching vessels (38, 40, 42) through the parent graft (12). Child grafts (not shown) may be deployed as described in reference to FIGS. 3I-3N.

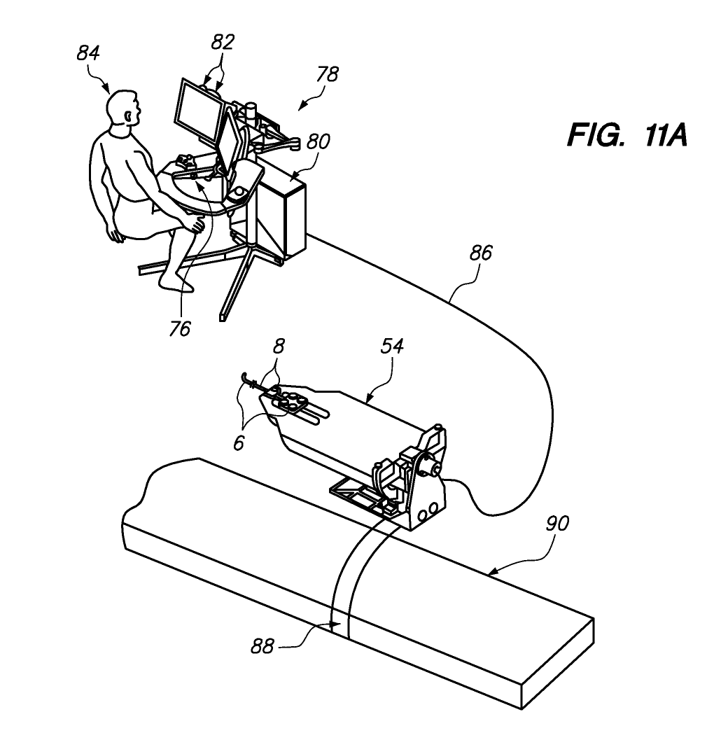

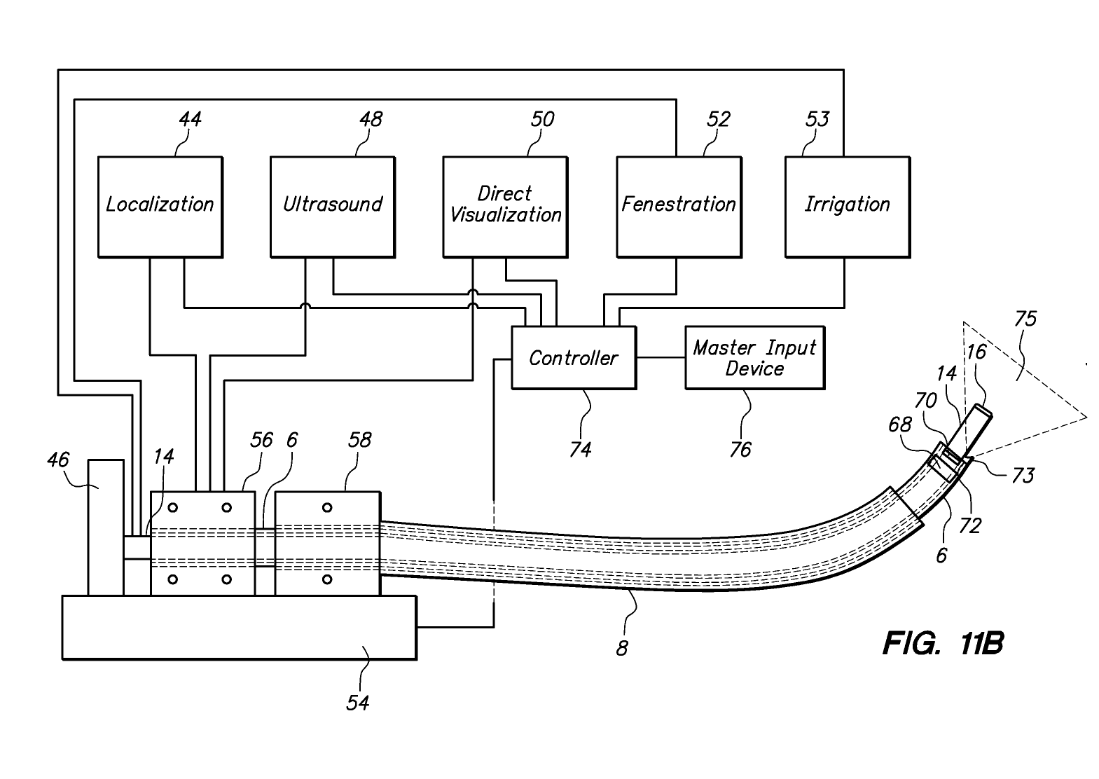

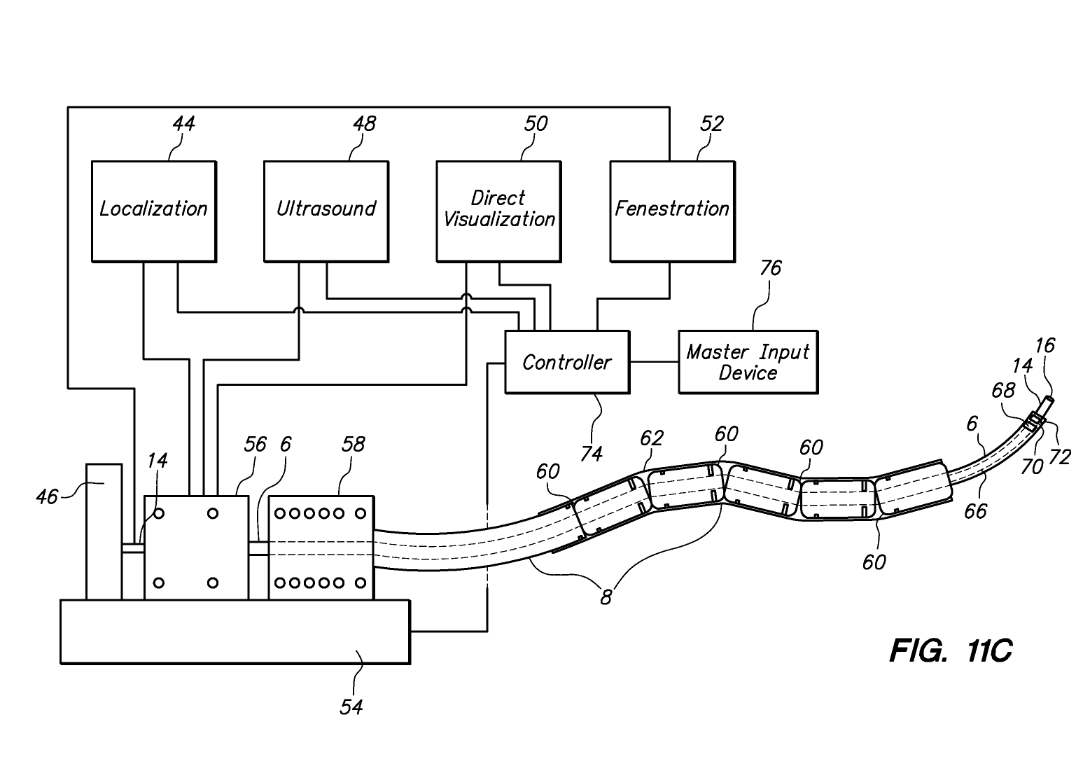

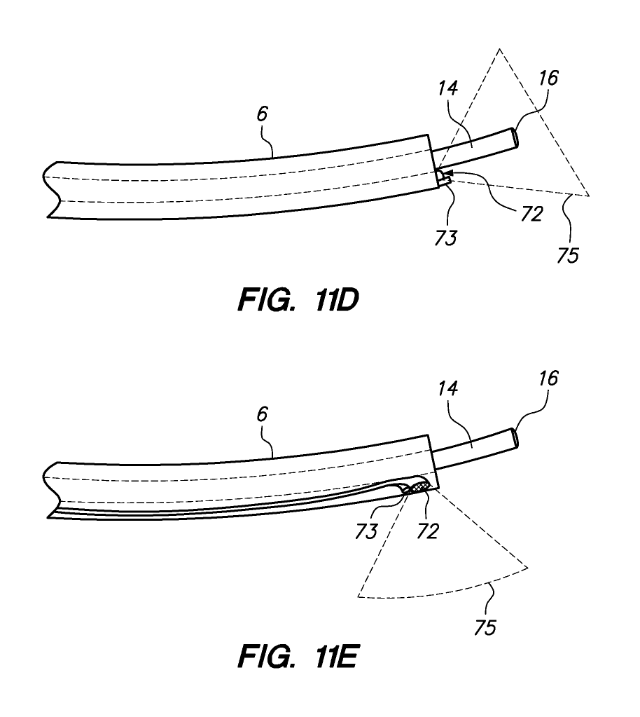

Referring to FIGS. 11A-11E, various aspects of systems and instruments configured for accomplishing in-situ graft fenestration as described above are depicted. Referring to FIG. 11A, a robotic catheter system is depicted having an operator workstation (78) wherein the operator (84) is able to observe images on one or more displays (82), and engage the system with, amongst other interfaces, a master input device (76) which is operatively coupled to a controller operated by a computer (80), the controller coupled to an instrument driver (54) by an electrical connection (86) such as a composite cable, and configured to cause motors within the instrument driver to induce controllable movements of the inner (6) and outer (8) steerable sheaths removably coupled to the instrument driver (54). The instrument driver (54) may be mounted above an operating table (90) utilizing a setup structure (88). Such a system is described in detail in the aforementioned incorporated by reference applications and is available from Hansen Medical, Inc., of Mountain View, Calif.

Referring to FIG. 11B, a variation of the system depicted in FIG. 11A is illustrated in partial diagrammatic view. Referring to FIG. 11B, inner (6) and outer (8) steerable sheaths are removably coupled to an instrument driver (54) utilizing interface structures (56, 58) and remotely steerable through manipulation of the master input device (76), which sends desired movement commands to the controller (74). A force sensing subsystem (46) is coupled to the instrument driver (54), and is operatively coupled to the proximal end of a fenestration instrument (14) which has been threaded through the working lumen of the inner sheath (6) to expose its distal end and a fenestration element (16) coupled thereto adjacent the distal end of the inner sheath instrument (6). The force sensing system (46) may be configured to sense forces applied to the distal aspects of the instrument to which it is coupled, here the fenestration probe (14), by utilizing oscillating, or “dithering”, motion of such probe (14) relative to the partially-surrounding inner sheath instrument (6), as described in the aforementioned incorporated by reference documents. A fenestration system (52), such as a current or power supply, is operably coupled to and commanded by the controller (74), and is operably coupled via an electrical lead to the fenestration element (16) at the distal end of the fenestration instrument (14). Preferably an operator at a master input device or other user interface may selectably command activation and deactivitation of the fenestration element through the controller (74). In irrigation system (53), such as a fluid reservoir and pump system, may be operably coupled to and commanded by the controller (74), and operably coupled via a tubing lead to the fenestration instrument (14) and/or inner (6) or outer (8) sheath instruments, depending upon the configuration at hand. In the embodiment depicted in FIG. 11B, irrigation fluid is directed, via an irrigation lumen formed into the inner sheath instrument, to a distal flush port (73) positioned to flush opaque fluids, such as blood, out of the forward-oriented field of view (75) of a direct visualization element (72), such as an optical fiber bundle image capture system or chip-based image capture device. In other words, the flush port is positioned to allow for controllable flushing of the field of view (75) of the visualization element (72). As described in the aforementioned incorporated-by-reference applications, the controller may be utilized to maintain “instinctiveness” between observed images on the system displays and the coordinate system of the master input device; the orientation transformations relating these subsystems may be automatically adjusted by the controller, or manually adjusted by the operator, to maintain instinctiveness and driveability of the pertinent steerable structures given the master input device and available image and navigation data through the user interface. Referring to FIGS. 11D and 11E, closer diagrammatic views of such structures are depicted. Referring to FIG. 11D, a direct visualization element (72) with a forward oriented field of view (75) may be positioned adjacent a forward-oriented flush port (73). Referring to FIG. 11E, a direct visualization element (72) with a side-oriented field of view (75) may be positioned adjacent a side-oriented flush port (73).

Referring back to the system of FIG. 11B, also coupled to the distal aspect of the inner sheath instrument (6) are a localization sensor (70) and an ultrasound transducer (68). The localization sensor (70) preferably is operably coupled with an external localization system (44), which may be operably coupled to the controller (74) to assist in navigating and locating the localization sensor (70) in three dimensional space. Suitable localization sensors and systems utilize electromagnetic flux measurements, potential different measurements, impedance measurements, fiber-Bragg techniques, and the like to determine location information, and are available from suppliers such as Biosense Webster, Inc. and St. Jude Medical Inc. The ultrasound transducer (68) may be operably coupled via an electrical lead to an external ultrasound system (48) to provide ultrasound images, amongst other feedback, such as time-of-flight proximity data, to the operator and controller. The direct visualization element (72) may be operably coupled, for example via fiber bundle or electrical lead, to a direct visualization system (50), which is configured to provide images for the operator to utilize at an operator workstation for navigation and other uses; the image data may also be returned to the controller (74) for assistance in operating the electromechanically-steerable sheaths (6, 8) and safely navigating them relative to other objects.

Referring to FIG. 11C, a system similar to that depicted in FIG. 11B is illustrated. In the embodiment of FIG. 11C, the outer sheath instrument (8) is reinforced by a controllably lockable spine, as described in the aforementioned and incorporated by reference Ser. No. 12/398,763 application. A series of lockable spine elements (60) are configured to be steerable and controllably lockable relative to each other. A sleeve (62) may at least partially encapsulate the lockable section to prevent pinch points and provide a smooth surface for tissue engagement. A controller (74) may be configured to not only drive the instrument driver (54) and thereby actuate the instruments (6, 8, and in some variations 14), but also to coordinate information such as commands from the user coming from a master input device (76), as well as data from localization, ultrasound, direct visualization, and fenestration systems associated with the instruments. For example, in the depicted embodiment, the distal end of the inner guide instrument comprises a localization sensor (70), an ultrasound transducer (68) configured to have a field of view positioned to capture images and data pertinent to nearby structures such as fenestrations and branching vessel intersections, a direct visualization imaging element (70), such as a fiber bundle or digital imaging chip, as well as a vacuum lumen (not shown).

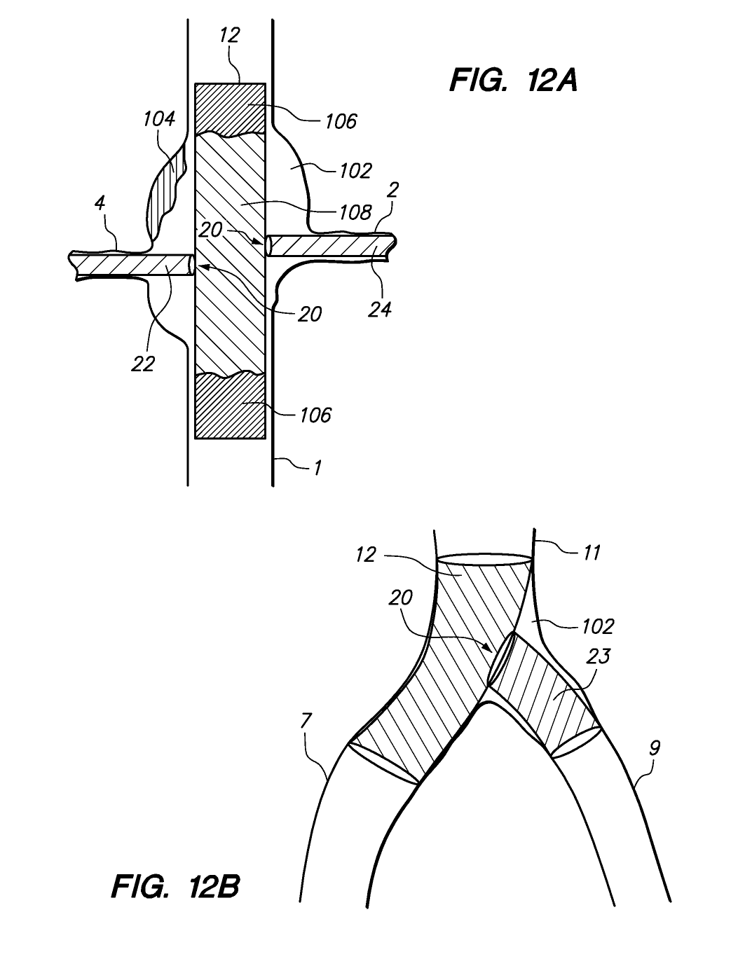

Referring to FIGS. 12A and 12B, the subject technology may be utilized in challenging anatomical and clinical situations which often do not present regular or homogeneous geometries or tissue mechanical properties. For example, referring to FIG. 12A, an aorta (1) is depicted with an irregularly-shaped aneurysm (102) and a plaque (104). A system such as those depicted in FIGS. 11A-11E may be utilized to install a graft assembly (12, 22, 24) as depicted in FIG. 12A. Also illustrated in FIG. 12A is the notion that a graft may be intentionally nonhomogeneous. The main parent graft (12) of the embodiment depicted in FIG. 12A has a middle region (108) which is configured to allow some perfusion of blood across its walls before fenestration has been accomplished, while the outer regions (106) are configured to not allow perfusion to avoid what may be known as “endoleaks” at the boundaries of the graft (12) where it significantly interfaces the aneurysm/aorta. Referring to FIG. 12B, the inventive technology may similarly be applied in a bifurcated lumen scenario, such as the depicted bronchial (7, 9,11) bifurcation of the lungs, wherein a parent graft (12) is first installed, then fenestrated (20) to accommodate installation of a child graft (23). Such anatomy may also be quite challenging, with irregularities, aneurysm-like geometries (102), etc.

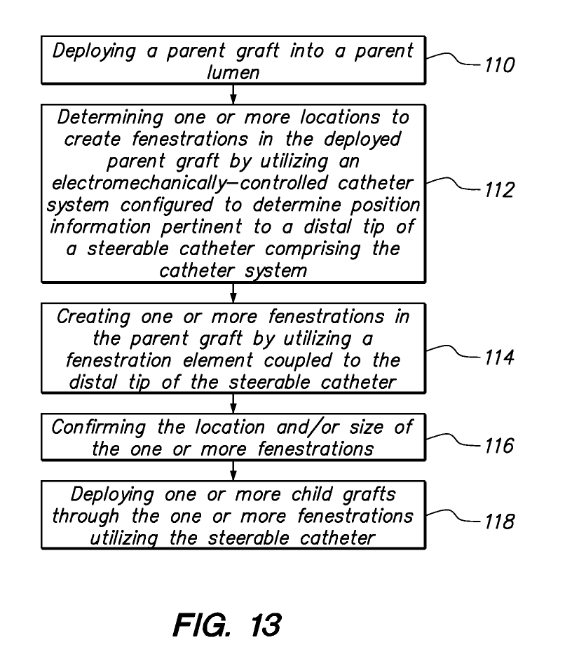

Referring to FIG. 13, a method for deploying a graft in accordance with the subject technology is illustrated. A parent graft is deployed into a parent lumen (110); one or more locations for fenestration are determined; this may be accomplished utilizing an electromechanically-controlled catheter system configured for determining position of one of more points along such catheter system (112); one or more fenestrations may be created in the parent graft by utilizing a fenestration element coupled to the distal tip of the steerable catheter (114); the location and/or size of the one or more fenestrations may be confirmed (116), for example using ultrasound (direct imaging or Doppler for flow-through), direct visualization, localization, inverse kinematics to localize the tip of the robotic instruments, etc; one or more child grafts may be deployed through the one or more fenestrations utilizing the steerable catheter (118). It is worth noting that while several of the depicted embodiments have a fenestration element coupled to a fenestration probe, such fenestration element may be coupled to any one of the elongate instruments described herein, and variations of the procedures and systems utilized with such hardware variations.

While multiple embodiments and variations of the many aspects of the invention have been disclosed and described herein, such disclosure is provided for purposes of illustration only. For example, wherein methods and steps described above indicate certain events occurring in certain order, those of ordinary skill in the art having the benefit of this disclosure would recognize that the ordering of certain steps may be modified and that such modifications are in accordance with the variations of this invention. Additionally, certain of the steps may be performed concurrently in a parallel process when possible, as well as performed sequentially. Accordingly, embodiments are intended to exemplify alternatives, modifications, and equivalents that may fall within the scope of the claims.

1. A robotic system for deploying a medical lumen graft, comprising:
A. a remotely steerable flexible instrument having proximal and distal ends and a graft fenestration element coupled to its distal end, the graft fenestration element configured to controllably create a fenestration through a wall of a deployed graft;
B. a controller in communication with a master input device; and
C. an instrument driver operatively coupled to the controller and the proximal end of the flexible instrument, the instrument driver configured to cause controlled steering movement of the flexible instrument in accordance with input signals received by the controller from the master input device.

2. The system of claim 1, wherein the graft fenestration element comprises a resistive element configured to heat to a cutting temperature upon application of a current to said resistive element.

3. The system of claim 2, wherein the resistive element comprises a wire loop.

4. The system of claim 3, wherein the wire loop comprises nichrome material.

5. The system of claim 1, wherein the graft fenestration element comprises a non-resistive discrete heat source.

6. The system of claim 5, wherein the non-resistive discrete heat source dissipates energy from a laser light source or an ultrasound transducer source.

7. The system of claim 1, wherein the graft fenestration element comprises a mechanical fenestration tip.

8. The system of claim 7, wherein the mechanical fenestration tip comprises a corkscrew tip or a mechanical dilation tip.

9. The system of claim 1, wherein the flexible instrument defines a lumen along the length of the flexible instrument.

10. The system of claim 9, further comprising a vacuum element coupled to the flexible instrument and configured to controllably provide vacuum through the lumen to assist in engagement of the flexible instrument with other nearby structures.

11. The system of claim 9, wherein the lumen is configured to facilitate controllable passage of a branch lumen graft through said lumen.

12. The system of claim 1, further comprising an elongate sheath instrument having a base, distal end portion, and a lumen through which the instrument is coaxially disposed, the instrument driver further comprising a sheath instrument interface operatively coupled to the sheath instrument base.

13. The system of claim 11, wherein the elongate sheath instrument comprises a controllably lockable spine.

14. The system of claim 9, wherein the lumen is a working lumen configured to accommodate elongate instruments inserted therethrough.

15. The system of claim 14, further comprising a force sensing apparatus coupled to the instrument driver and configured to sense forces applied distally to instruments inserted through the working lumen.

16. The system of claim 1, further comprising a localization sensor coupled to the flexible instrument, the localization sensor configured to determine the spatial position of at least a portion of the flexible instrument.

17. The system of claim 16, wherein the localization sensor is selected from the group consisting of an electromagnetic localization sensor, a potential difference localization sensor, and a fiber-bragg localization sensor.

18. The system of claim 1, further comprising an ultrasound transducer coupled to the distal end portion of the flexible instrument, the ultrasound transducer having a field of view configured to be able to capture reflected sound information pertinent to a side branch vessel location and geometry.

19. A method for deploying a lumen graft, comprising:
a. deploying a parent lumen graft in a parent lumen;
b. determining one or more locations to create fenestrations in the deployed parent lumen graft by utilizing a electromechanically-controlled catheter system configured to determine position information pertinent a distal tip of a steerable catheter comprising the catheter system; and
c. creating one or more fenestrations in the parent lumen graft by utilizing a fenestration element coupled to the distal tip of the steerable catheter.

20. The method of claim 19, wherein determining locations comprises utilizing a kinematic relationship established for the steerable catheter to determine a position of the distal tip of said steerable catheter.

21. The method of claim 19, wherein determining locations comprises utilizing a localization system selected from the group consisting of an electromagnetic localization sensing system, a potential difference localization sensing system, and a fiber-bragg localization sensing system.

22. The method of claim 19, wherein the fenestration element comprises a resistive heating element, and wherein creating fenestrations comprises controllably providing electrical current to said resistive heating element.

23. The method of claim 19, wherein the fenestration element comprises a non-resistive discrete heat source selected from the group consisting of a laser light source or an ultrasound transducer source, and wherein creating fenestrations comprises controllably providing electrical current to said source.

24. The method of claim 19, wherein the fenestration element comprises a mechanical fenestration tip selected from the group consisting of a corkscrew tip and a mechanical dilation tip, and wherein creating fenestrations comprises advancing such tip through a wall of the lumen graft.

25. The method of claim 19, further comprising applying vacuum through a lumen defined through the steerable catheter to encourage coupling of said catheter to other nearby structures.

26. The method of claim 19, further comprising confirming the location or size of the one or more fenestrations.

27. The method of claim 26, wherein confirming comprises utilizing a kinematic relationship established for the steerable catheter to determine a position of the distal tip of said steerable catheter when positioned adjacent the one or more fenestrations.

28. The method of claim 26, wherein confirming comprises utilizing a localization sensor disposed at least in part at the distal tip of the steerable catheter, the localization sensor selected from the group consisting of an electromagnetic localization sensor, a potential difference localization sensor, and a fiber-bragg localization sensor.

29. The method of claim 26, wherein confirming comprises utilizing an ultrasound transducer coupled to the distal portion of the steerable catheter to capture an image of the one or more fenestrations.

30. The method of claim 26, wherein confirming comprises utilizing a contrast agent disbursal adjacent the location of the one or more fenestrations, along with fluoroscoping imaging, to locate and size the one or more fenestrations.

31. The method of claim 26, wherein confirming comprises utilizing a force sensor to locate and size the or more fenestrations.

32. The method of claim 19, further comprising deploying a child lumen graft through one of the one or more fenestrations utilizing the steerable catheter.

33. The method of claim 32, further comprising utilizing an inflatable balloon element to mechanically seat the child lumen graft relative to the parent lumen graft.

34. The method of claim 19, further comprising confirming the location or size of the one or more child lumens intersecting with the parent lumen.

35. The method of claim 34, wherein confirming comprises utilizing a kinematic relationship established for the steerable catheter to determine a position of the distal tip of said steerable catheter when positioned adjacent the one or more fenestrations.

36. The method of claim 34, wherein confirming comprises utilizing a localization sensor disposed at least in part at the distal tip of the steerable catheter, the localization sensor selected from the group consisting of an electromagnetic localization sensor, a potential difference localization sensor, and a fiber-bragg localization sensor.

37. The method of claim 34, wherein confirming comprises utilizing an ultrasound transducer coupled to the distal portion of the steerable catheter to capture an image of the one or more fenestrations.

38. The method of claim 34, wherein confirming comprises utilizing a contrast agent disbursal adjacent the location of the one or more fenestrations, along with fluoroscoping imaging, to locate and size the one or more fenestrations.

39. The method of claim 34, wherein confirming comprises utilizing a force sensor to locate and size the or more fenestrations.
